(19) United States
(12) Patent Application Publication
(10) Pub. No.: US 2026/0168989 A1
(43) Pub. Date: Jun. 18, 2026

(54) SYSTEMIC ORGANOID SIGNATURES CHIP AND METHODS OF USE

(71) Applicant: The Johns Hopkins University, Baltimore, MD (US)

(72) Inventors: Julia B. Patrone, Ellicott City, MD (US); Andrea C. Timm, Ellicott City, MD (US); Claire M. Bell, Crownsville, MD (US); Chad N. Weiler, Columbia, MD (US); Neil C. Momsen, Ellicott City, MD (US)

(21) Appl. No.: 19/343,327

(22) Filed: Sep. 29, 2025

Related U.S. Application Data
(60) Provisional application No. 63/733,700, filed on Dec. 13, 2024.

(51) Int. Cl. G01N 33/50; G06T 7/00

(52) U.S. Cl. CPC G01N 33/5088; G06T 7/0014; G01N 2500/10; G06T 2207/30024

(57) ABSTRACT

A method of evaluating an effect of at least one agent on an organoid is disclosed herein. The method includes providing the organoid in a cell culture or microfluidic platform, contacting the organoid with at least one agent, acquiring a plurality of images of the organoid and a control organoid, and analyzing the plurality of images for a change of at least one characteristic of the organoid, wherein the change of the at least one characteristic is indicative of a disease or a disorder, and wherein the analyzing comprises comparing the organoid to the control organoid to determine the change of the at least one characteristic. The method also includes assessing the effect of the at least one agent at least in part on the change of the at least one characteristic of the organoid.

### CROSS-REFERENCE TO RELATED APPLICATIONS

The present application claims benefit under 35 U.S.C. § 119(e) to U.S. Provisional Ser. No. 63/733,700 filed on Dec. 13, 2024, which is hereby incorporated by reference in its entirety.

### TECHNICAL FIELD

The present disclosure relates to methods of identifying and characterizing pathogens by utilizing an organoid model system.

### BACKGROUND

The first sign of an infectious disease outbreak is clinical case reporting. At present, there is no way to rapidly predict the pathogenicity and pandemic potential of an unknown microbe or virus. One strong indicator of a highly pathogenic virus is tissue tropism, or the ability of a pathogen to infect and replicate in a given tissue, e.g., a preferred tissue for the pathogen.

There is currently a significant gap between the identification of a pathogen during an outbreak and the prediction of its ability to cause severe disease in humans. Current methods for prediction are based on genomic sequencing and taxonomy paired with clinical morbidity and mortality reporting. This gap limits our ability to rapidly respond to disease outbreaks, whether naturally occurring or resulting from intentional release.

Thus, methods for predicting pathogenicity that rapidly characterize a pathogen are needed to better respond to infectious disease outbreaks.

### SUMMARY

In one aspect, a method of evaluating an effect of at least one agent on an organoid is disclosed. The method includes providing the organoid in a cell culture or microfluidic platform; contacting the organoid with at least one agent; acquiring a plurality of images of the organoid and a control organoid; analyzing the plurality of images for a change of at least one characteristic of the organoid, wherein the change of the at least characteristic is indicative of a disease or a disorder, wherein the analyzing comprises comparing the organoid to the control organoid to determine the change of the at least one characteristic; and assessing the effect of the at least one agent at least in part on the change of the at least one characteristic of the organoid.

In another aspect, a method for identifying a therapeutic agent for preventing or treating a disease or disorder is disclosed. The method includes providing a diseased organoid in a cell culture or microfluidic platform; administering the therapeutic agent to the diseased organoid generating a treated organoid; acquiring a plurality of images of the treated organoid and a control organoid; analyzing the plurality of images for a change of at least one characteristic of the treated organoid, wherein the change of the at least one characteristic is indicative of the suitability of the therapeutic agent for preventing or treating the disease or disorder, wherein the analyzing comprises comparing the treated organoid to the control organoid to determine the change of the at least one characteristic; and identifying a candidate therapeutic agent for preventing or treating the disease or disorder at least in part based on the change of the at least one characteristic of the organoid.

### BRIEF DESCRIPTION OF THE DRAWINGS

The patent or application file contains at least one drawing executed in color and at least one color photograph. Copies of this patent or patent application publication with color drawing(s) and color photograph(s) will be provided by the Office upon request and payment of the necessary fee. At least one photograph has been included in the application because the at least one photograph is the only practicable medium for illustrating the claimed invention.

FIG. 1 depicts a schematic diagram of a systemic organoid signatures chip in accordance with embodiments described herein.

FIG. 2A depicts the differential staining that was used to show gut organoid model development (specifically goblet cells and enteroendocrine cells) in accordance with embodiments described herein.

FIG. 2B depicts images of kidney tubuloids growing in cell culture media in accordance with embodiments described herein.

FIG. 3 depicts gut organoids infected with low and high MOI (multiplicity of infection) of adenovirus type 41(Ad41 ) in accordance with embodiments described herein. Infection levels were assayed after 120 hours by qPCR.

FIG. 4 depicts examples of organoid images by brightfield microscope, including a summation of images, and a differential phase contrast (DPC) image in accordance with embodiments described herein.

FIG. 5 depicts confluent gut organoids in cell culture infected by adenovirus or as an uninfected control in accordance with embodiments described herein.

FIG. 6A depicts a brightfield image of an organoid taken at 72 hours post infection in accordance with embodiments described herein.

FIG. 6B depicts a DPC image of an organoid taken at 72 hours post infection in accordance with embodiments described herein.

FIG. 7A depicts a quantitative phase image (QPI) of an uninfected organoid in accordance with embodiments described herein. Regions of interest (ROI) in the background and organoid were averaged and compared (boxes).

FIG. 7B depicts a quantifiable phase image (QPI) of an infected organoid in accordance with embodiments described herein. Regions of interest (ROI) in the background and organoid were averaged and compared (boxes).

FIG. 8 depicts a graph comparing the difference in phase between infected and uninfected organoids over 72 hours post infection in accordance with embodiments described herein. ROIs for each sample were averaged and compared over the 72 hours.

FIGS. 9A and 9B depict example methods 1100A and 1100B in accordance with at least one of the disclosed embodiments.

FIG. 10 depicts an example computer system and apparatus in accordance with embodiments described herein.

Various embodiments are described in detail below with reference to the accompanying drawings, wherein like reference numerals represent like elements, and wherein descriptions of like elements may not be repeated for every embodiment, but may be considered to be the same if previously described herein.

The figures provided herein are for illustrative purposes and may not be to scale. Variations in dimensions, proportions, and configurations may exist between the figures and the actual embodiments. The figures are intended to facilitate understanding of the embodiments and should not be construed as limiting the scope of the disclosure.

### DETAILED DESCRIPTION

Studying infections in multiple human organoid models may be predictive of the potential for a pathogen to cause serious disease in people. A multi-organoid model may be used to screen pathogens using rapid, non-destructive imaging approaches designed to predict pathogenicity. In some embodiments, the multi-organoid model may be used to screen therapeutic agents for the treatment of pathogenic infection. In some embodiments, the multi-organoid model may be used to screen therapeutic agents for the treatment of diseases or disorders.

A method has been developed to differentiate pathogens (like viruses) simply by: 1) infecting a complex cellular model, 2) rapidly collect brightfield images (no staining required) over time, and 3) analyzing them to determine whether they are infected or not. Preliminary results suggest that this approach is sufficient to differentiate between infected and uninfected colonoid and kidney organoids and may enable use of deep learning models. Different types of organoids and viruses may be used to develop these analytic models.

A quantitative phase imaging microscope setup may be used to facilitate measurement of refractive index and organoid morphology as potential additional signatures of infection that could be incorporated into future iterations of a deep learning model.

### Organoids

The term “organoid” may refer to an in vitro collection of cells that form 3D structures, which resemble their in vivo counterparts. Use of organoids for the methods as described herein may be used for in vitro methods.

In some embodiments, the organoids may be mammalian organoids, for example human organoids, that may be derived from cells taken from a mammal. The mammal may be any mammal of interest, for example a human or a mouse. In some embodiments the organoids are non-human. A preferred organoid may be human.

In some embodiments, the organoids of the assay may be gut organoids, e.g., primary cells from the colon or intestine. However, the organoids may be derived from other tissues of the mammal.

In some embodiments, the organoids may be derived from induced pluripotent stem cells (iPSCs).

Organoids may comprise a lumen surrounded by cells. The cells of an organoid may be the same cell, or may comprise multiple different types of cells. For example, a gut organoid may comprise goblet cells, enteroendocrine cells, and/or enterocytes. An organoid may comprise stem cells, such as multipotent stem cells specific to the type of tissue the organoid is based (e.g., intestinal stem cells).

The term “resembles” may indicate that the organoid has genetic and phenotypic characteristics that allow it to be recognized by the skilled person as being from or associated with a particular tissue type, such as intestinal tissues. The organoid may be genetically and phenotypically identical or partially identical to the in vivo tissue cell type.

In some embodiments, the organoids may be “normal” organoids, that is organoids may mimic the genotype and phenotype of the in vivo tissue cell types. In some embodiments, the organoids may be “disease” organoids. Diseased organoids may mimic the in vivo disease genotype and phenotype.

Organoids may be generated and grown in a cell culture media, such as, but not limited to the cell culture media similar cells would require for growth in suspension. For example, cell culture media may be, but not limited to, Minimum Essential Medium (MEM), Dulbecco's Modified Eagle Medium (DMEM), and RPMI-1640 medium. The cell culture media may be supplemented with nutrients and other components for the growth of the specific cell type used to generate the organoid.

### Methods of Evaluating an Organoid

As discovered by the inventors, the methods provided herein may be for evaluating an effect of a pathogen and/or a chemical/biological agent on an organoid, the method comprising: providing the organoid in a cell culture or microfluidic platform; contacting the organoid with at least one pathogen and/or chemical/biological agent; acquiring, with quantitative phase imaging, a plurality of images of the organoid and a control organoid; analyzing the plurality of images for a change of at least one characteristic of the organoid, wherein the change of the at least one characteristic is indicative of a disease or a disorder, wherein the analyzing comprises comparing the organoid to the control organoid to determine the change of the at least one characteristic; and assessing the effect of the at least one pathogen and/or chemical agent at least in part on the change of the at least one characteristic of the organoid.

An organoid used for testing may be a normal organoid which has been infected with a pathogen resulting in an infected organoid. The pathogen may include, but is not limited to viruses, bacteria, fungi, or parasites. Examples of viruses may include influenza viruses, rhinoviruses, coronaviruses, human immunodeficiency virus (HIV), and hepatitis viruses.

Pathogenicity of a pathogen may refer to the ability of the pathogen to infect and cause illness in a host, such as humans. Virulence refers to the severity and intensity of the disease caused by the pathogen. Virulence includes measuring the extent of damage, cellular and whole body, symptoms and mortality caused by the pathogen.

Mutations in the genome of the pathogen may cause a strain of a pathogen be more or less virulent than the precursor pathogen. Some pathogens have high mutation rates causing the pathogenicity and virulence measures to change for each generation of the pathogen. For example, influenza causes the flu in humans and each flu season commonly sees a new strain(s) of the virus due to the high mutation rate. Any mutation in the pathogen has the potential to affect the susceptibility of the pathogen to the known treatments. For example, new strains of influenza may require a different vaccine compared to the precursor.

An organoid used for testing may be a normal organoid which has been treated with a chemical compound. A chemical compound may include, but is not limited to a treatment for a pathogen, a treatment for a disease or a disorder, such as cancer or irritable bowel syndrome (IBS). The chemical compound may be administered to a normal organoid and/or an infected organoid, and/or a diseased organoid.

A control organoid to an organoid infected with a pathogen may comprise an organoid that has not been infection by the pathogen or an organoid infected with a known pathogen (e.g., a pathogen with known pathogenicity and/or virulence measurements).

A control organoid to an organoid treated with a chemical compound may comprise an organoid that has not been treated with the chemical compound.

A control organoid to an organoid infected with a pathogen and treated with a therapeutic agent may comprise an organoid not infected with the pathogen, an organoid treated with the therapeutic agent, an organoid infected with the pathogen, or an organoid infected with a known pathogen.

A control organoid for a diseased organoid treated with a therapeutic agent (i.e., treated organoid) may comprise a diseased organoid not treated with the therapeutic agent, a normal organoid, or a normal organoid treated with the therapeutic agent.

In some embodiments, the characteristic may be a physical, cellular, and/or molecular characteristic. In some embodiments, the characteristic may be size. In some embodiments, the characteristic may be refractive index. In some embodiments, the characteristic may be degree of differentiation. In some embodiments, the characteristic may be the relative proportions of different cell types. In some embodiments, the characteristic may be the shape or sphericity. In some embodiments, the characteristic may be the level of expression of a particular gene, genes, protein, or proteins. In some embodiments, the characteristic may be the level of production of a metabolite, biological product, or chemical product. In some embodiments, the characteristic may be the rate of consumption or uptake of a metabolite, a biological product, or a chemical product.

In some embodiments, the images of the organoid and the control organoid may be two-dimensional (2D) images. In some embodiments, the images of the organoid and the control organoid may be three-dimensional (3D) images. In some embodiments, the images of the organoid and the control organoid may be acquired using a label-free method. In some embodiments, the label free method may be brightfield, darkfield, differential interference contrast, quantitative phase imaging (QPI), birefringence, optical coherence tomography, gradient light interference microscopy, polarization light microscopy, rotating coherent scattering. In some embodiments, the images of the organoid and the control organoid may be acquired using a labeling method.

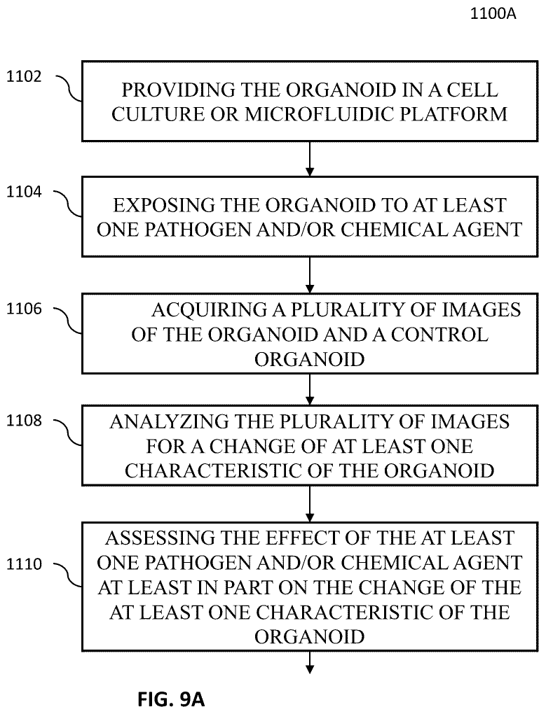

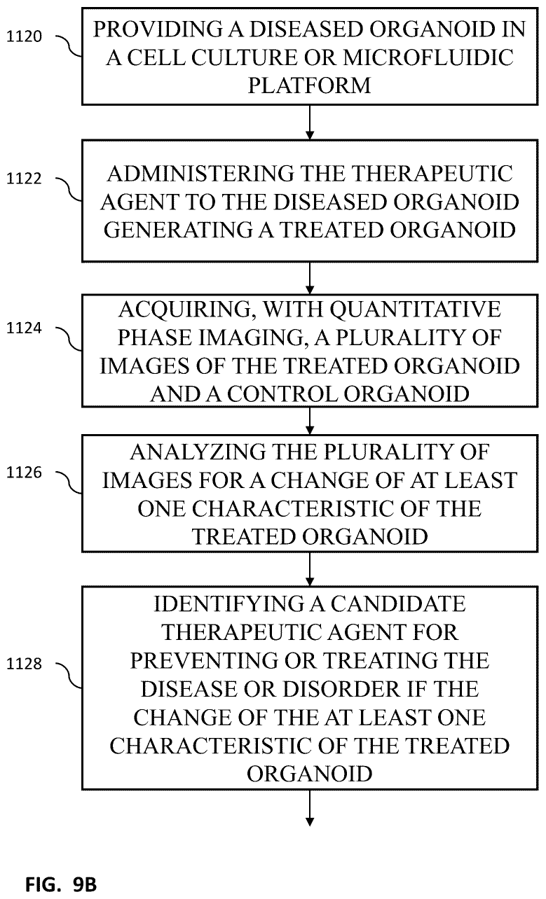

FIG. 9A illustrates an example method 1100A of identifying and characterizing pathogens by utilizing an organoid model system. While an order of operations is indicated in FIG. 9A for illustrative purposes, the timing and ordering of such operations may vary where appropriate without negating the purpose and advantages of the examples set forth in detail herein.

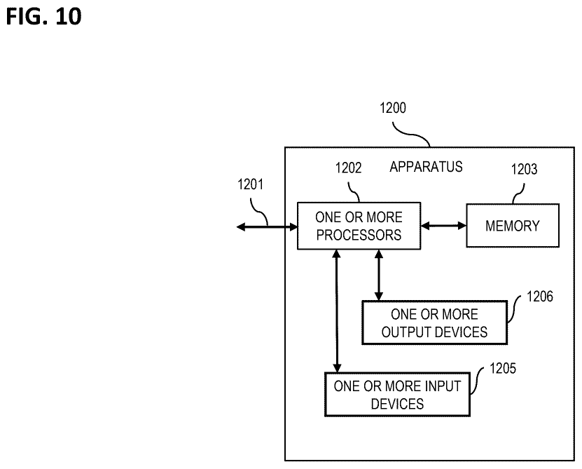

Method 1100A may be implemented by a computer apparatus. The computer apparatus may be an instance of apparatus 1200 shown in FIG. 12. While FIG. 11A will be discussed in the context of FIG. 10, this is for clarity of explanation purposes only and the method described in FIG. 10 should not be considered limited by the components illustrated in FIG. 10. Method 1100A may be implemented by a processor, such as processor 1202 of FIG. 10.

Method 1100A, at 1102, may include processor 1202 providing the organoid in a cell culture or microfluidic platform. Method 1100A, at 1104, may include processor 1202 contacting the organoid with at least one pathogen and/or chemical agent. Method 1100A, at 1106, may include processor 1202 acquiring a plurality of images of the organoid and a control organoid. Method 1100A, at 1108, may include processor 1202 analyzing the plurality of images for a change of at least one characteristic of the organoid. Method 1100A, at 1110, may include processor 1202 assessing the effect of the at least one pathogen and/or chemical agent at least in part on the change of the at least one characteristic of the organoid. In some embodiments, the effect of the at least one pathogen and/or chemical agent may be assessed with a neural network. For example, the neural network may be trained to predict the effect of the at least one pathogen when provided with the plurality of images of an uninfected organoid and an infected organoid. In some embodiments, step 1110 may be performed with a trained machine learning model. For example, the machine learning model may be trained with a plurality of images of organoids appropriately labeled as infected or uninfected with respect to a pathogen. Further, the machine learning model may be trained to predict the effect of a pathogen when provided with images of an infected or uninfected organoid. In some embodiments, the machine learning model may be a convolutional neural network, as further discussed below. In some embodiments, the machine learning model may be trained using supervised or unsupervised learning, as further discussed below.

As discovered by the inventors, the methods described herein may be used to identify a therapeutic agent for preventing or treating a disease or disorder, the method comprising: providing a diseased organoid in a cell culture or microfluidic platform; administering the therapeutic agent to the diseased organoid generating a treated organoid; acquiring, with quantitative phase imaging, a plurality of images of the treated organoid and a control organoid; analyzing the plurality of images for a change of at least one characteristic of the treated organoid, wherein the change of the at least one characteristic is indicative of the suitability of the therapeutic agent for preventing or treating the disease or disorder, wherein the analyzing comprises comparing the treated organoid to the control organoid to determine the change of the at least one characteristic; and identifying a candidate therapeutic agent for preventing or treating the disease or disorder if the change of the at least one characteristic of the treated organoid.

In some embodiments, the diseased organoid comprises an organoid infected with a pathogen. In some embodiments, the disease organoid comprises an organoid derived from diseased cells and resemble in vivo diseased tissues. The disease or disorder may include, but is not limited to cancer, irritable bowel syndrome (IBS), inflammatory bowel disease (IBD), Crohn's disease, fibrosis, cystic fibrosis, cardiac fibrosis, liver fibrosis, and kidney disease.

### Quantitative Phase Imaging (QPI)

The multilayered, three-dimensional (3D) nature of organoids can be challenging to study using traditional microscopy techniques, because of methods of preparing the samples can require fixation and/or labeling, which hinder real-time data to be acquired. Additionally, images gathered by many microscopy techniques result in two-dimensional (2D) images, which could limit resolution of the morphological changes of an organoid through the duration of an assay. Therefore, robust microscopy techniques are needed to better analyze morphological changes of an organoid after exposure to a pathogen or chemical agent.

Quantitative phase imaging (QPI) may be used to acquire 3D images of organoids and to study morphological changes of the organoids. QPI uses a microscope to measure the phase shift of light as it interacts with a sample, such as an organoid. The phase shift is related to the refractive index and morphology of the organoid.

A plurality of images from QPI may be acquired over time and analyzed at region of interest (ROI). The phase shift may be measured for each time point and compared to control organoid(s).

The measurements from the QPI may focus on one or more characteristics to be measured for comparison. For example, QPI may measure at least refractive index, thickness and dry mass. A change in one or more characteristics of the organoid being tested may indicate if the organoid is diseased compared to a normal organoid. A change in a characteristic may show a change in size of the organoid being tested. For example, the organoid may decrease, stay the same, or increase in size over time.

A change in a characteristic may show swelling of cells of the organoid. In some embodiments, the swelling of cells of the organoid increases compared to prior to contacting the organoid with the at least one pathogen and/or chemical agent.

In some embodiments, assessing the effect of the at least one pathogen and/or chemical agent comprises comparing the change of the at least one characteristic of the organoid to the control organoid. In some embodiments, the method may further include comparing the refractive index from the plurality of images of the organoid and/or the control organoid for at least 1 hour after contacting the organoid with at least one pathogen and/or chemical agent.

In some embodiments, the analyzing of the organoids may be for about 12 hours, about 24 hours, about 36 hours, about 48 hours, about 60 hours, about 3 days, about 4 days, about 5 days, about 6 days, about 1 week, or about 2 weeks after contacting the organoid with at least one pathogen and/or chemical agent. In some embodiments, the analyzing of the organoids may be done continuously (e.g., at or near the maximum acquisition rate of the technique).

As described herein, the methods may comprise cell culture or multifluidic platform comprising the organoids. The multifluidic platform may include individual wells or each condition being tested and analyzed.

FIG. 9B illustrates an example method 1100B of identifying and characterizing pathogens by utilizing an organoid model system. Method 1100B may be implemented by a computer apparatus. While an order of operations is indicated in FIG. 9B for illustrative purposes, the timing and ordering of such operations may vary where appropriate without negating the purpose and advantages of the examples set forth in detail herein.

The computer apparatus may be an instance of apparatus 1200 shown in FIG. 10. While FIG. 9B will be discussed in the context of FIG. 10, this is for clarity of explanation purposes only and the method described in FIG. 10 should not be considered limited by the components illustrated in FIG. 10. Method 1100B may be implemented by a processor, such as processor 1202 of FIG. 10.

Method 1100B, at 1120, may include processor 1202 providing a diseased organoid in a cell culture or microfluidic platform. Method 1100B, at 1122, may include processor 1202 administering the therapeutic agent to the diseased organoid generating a treated organoid. Method 1100B, at 1124, may include processor 1202 acquiring, with quantitative phase imaging, a plurality of images of the treated organoid and a control organoid. Method 1100B, at 1126, may include processor 1202 analyzing the plurality of images for a change of at least one characteristic of the treated organoid. Method 1100B, at 1128, may include processor 1202 identifying a candidate therapeutic agent for preventing or treating the disease or disorder if the change of the at least one characteristic of the treated organoid. In some embodiments, step 1126 may be performed with a trained machine learning model. For example, the machine learning model may be trained with a plurality of images of organoids appropriately labeled as infected or uninfected with respect to a pathogen. Further, the machine learning model may be trained to predict the effect of a pathogen when provided with images of an infected or uninfected organoid. In some embodiments, the machine learning model may be a convolutional neural network, as further discussed below. In some embodiments, the machine learning model may be trained using supervised or unsupervised learning, as further discussed below.

### Definitions

In order that the invention may be more readily understood, certain terms are first defined. In addition, it should be noted that whenever a value or range of values of a parameter are recited, it is intended that values and ranges intermediate to the recited values are also intended to be part of this invention.

As used herein and in the appended claims, the singular forms “a,” “an,” and “the” include plural reference unless the context clearly dictates otherwise. Thus, for example, reference to a “peptide” is a reference to one or more peptides and equivalents thereof known to those skilled in the art, and so forth.

The term “or” is used herein to mean, and is used interchangeably with, the term “and/or,” unless context clearly indicates otherwise.

The terms “about” and “approximately” are used herein to mean within the typical ranges of tolerances in the art. For example, “about” may be understood as about 2 standard deviations from the mean. According to certain embodiments, “about” means +10%. According to certain embodiments, “about” means +5%. When “about” is present before a series of numbers or a range, it is understood that “about” may modify each of the numbers in the series or range.

The term “at least” prior to a number or series of numbers (e.g. “at least two”) is understood to include the number adjacent to the term “at least”, and all subsequent numbers or integers that could logically be included, as clear from context. When “at least” is present before a series of numbers or a range, it is understood that “at least” may modify each of the numbers in the series or range.

As used herein, “up to” as in “up to 10” is understood as up to and including 10, e.g., 0, 1, 2, 3, 4, 5, 6, 7, 8, 9, or 10. The phrase “up to” may include integers and non-integers, e.g., 0, 0.1, 0.2, 0.3, 0.4, 0.5, 0.6, 0.7, 0.8, 0.9, or 1.

Ranges provided herein are understood to include all individual integer and non-integer values and all subranges within the ranges.

As used herein, the term “in combination with,” is not intended to imply that the therapy or the therapeutic agents must be administered at the same time and/or formulated for delivery together, although these methods of delivery are within the scope described herein. The therapeutic agents may be administered concurrently with, prior to, or subsequent to, one or more other additional therapies or therapeutic agents.

The term “treatment” refers to administering an active agent (e.g., a therapeutic agent) with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

The term “therapeutic” as used herein means a treatment and/or prophylaxis. A therapeutic effect is obtained by suppression, remission, or eradication of a disease state. As used herein, the term “therapeutic agent” may refer to, but is not limited to, a pharmaceutical agent, a pharmaceutical composition, a gene, a nucleic acid, a protein, a cell, a molecule, or a substance that can be used to treat, control, or prevent a disease, medical condition, or disorder. An effect of the therapeutic agent may include, but is not limited to, modification of the genome of a target cell or target cell population (e.g. “gene therapy”).

### Implementation Examples

### Example 1

Methods: Gut organoids derived from intestinal crypts and kidney tubuloids differentiated from induced pluripotent stem cells (iPSCs) were used in this work. Organoids were infected with Adenovirus and Influenza A. Post-infection, brightfield images were collected every 2-4 hours for several days using plate imagers equipped with live-cell environmental controls. Image data were split into training and testing sets. A convolutional neural network (i.e., ResNet-50) was then used to determine if brightfield images were sufficient to correctly classify infected and uninfected tissues.

Results: Qualitatively, differences in infection of different organoids with different viruses were seen, resulting in correct classification of images of infected and uninfected organoids. Work is ongoing to ensure overfitting the model is not occurring and to determine how soon after infection the model is able to correctly identify infection.

Conclusions: Pathogen-agnostic brightfield imaging data may be sufficient to train a neural network model to differentiate infected and uninfected organoids. Different types of organoids and viruses may be used to train this model.

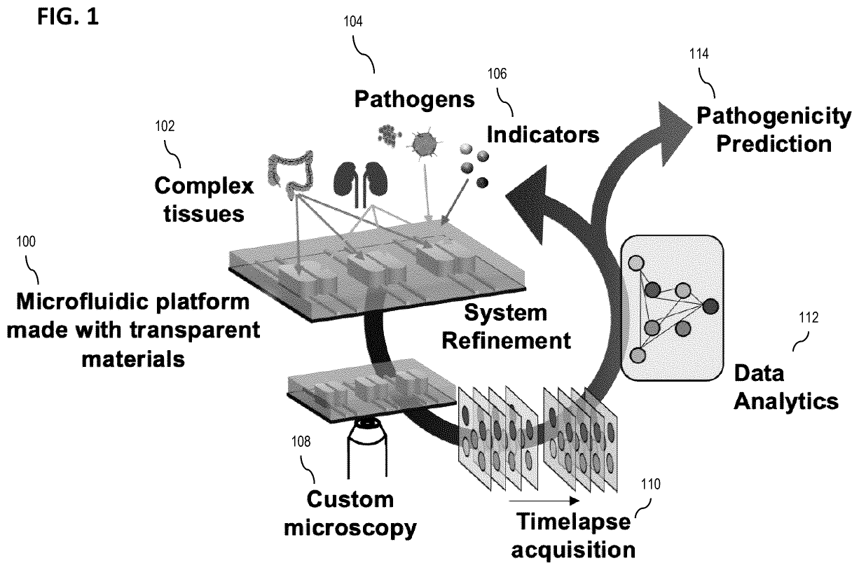

Hypothesis and Project Vision: Tissue tropism is one of the leading indicators of pathogenicity. The result of infections in advanced tissue culture and organoid models may be used as an indicator of tropism. High-throughput and high-content data may be extracted from these systems to train a computer vision model to differentiate highly pathogenic organisms from more benign pathogens with the goal of creating a pathogenicity prediction algorithm. The systemic organoid chip and this algorithm could then be used together to identify the next pandemic-potential strain before containment becomes intractable. A schematic diagram of a systemic organoid chip and methods of gathering timelapse images is shown in FIG. 1. A microfluidic platform 100 was made with transparent material. Complex tissues 102, such as organoids, are placed in microfluidic platform well. Pathogens 104 or indicators 106, such as chemical compositions or therapeutic agents, may be tested with the organoids. The microscope 108 was designed for quantitative phase imaging (QPI). Images may be retrieved over time at per-determined time intervals 110. Data analytics 112 may train or test a model neural network. Prediction 114, e.g., pathogenicity prediction or effectiveness of a therapeutic agent, may be the output of the system.

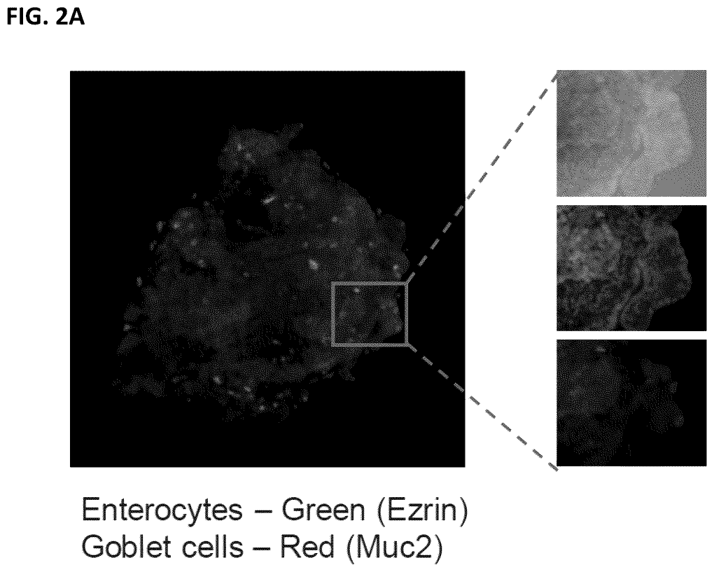

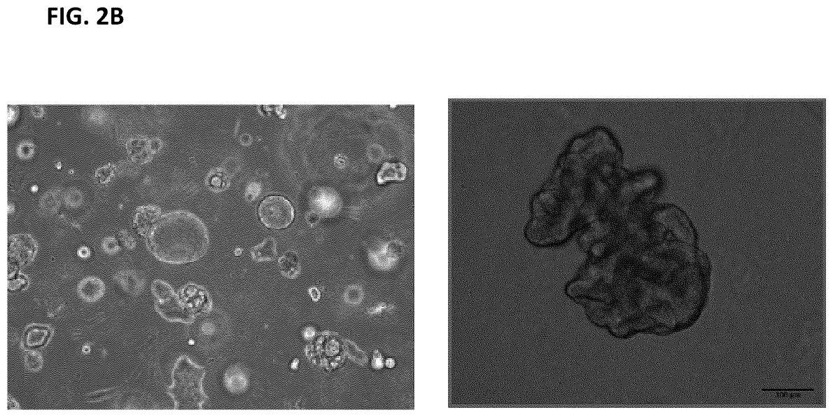

Organoid Model Development and Validation: Organoids from every major organ system may use this system. Gut organoids isolated from the human colon and kidney tubuloid organoids have been generated from induced pluripotent stem cells (iPSCs). Gut organoids were chosen because ingestion is a route of exposure to pathogens. The kidney tubuloid model was chosen because renal tropism is a particularly strong indicator of a highly pathogenic infection. FIGS. 2A-2B show generation of organoids as determined by staining to indicate the presence of goblet and enteroendocrine cells (Gut organoids, FIG. 2A) and microscopy of developing kidney organoids (FIG. 2B).

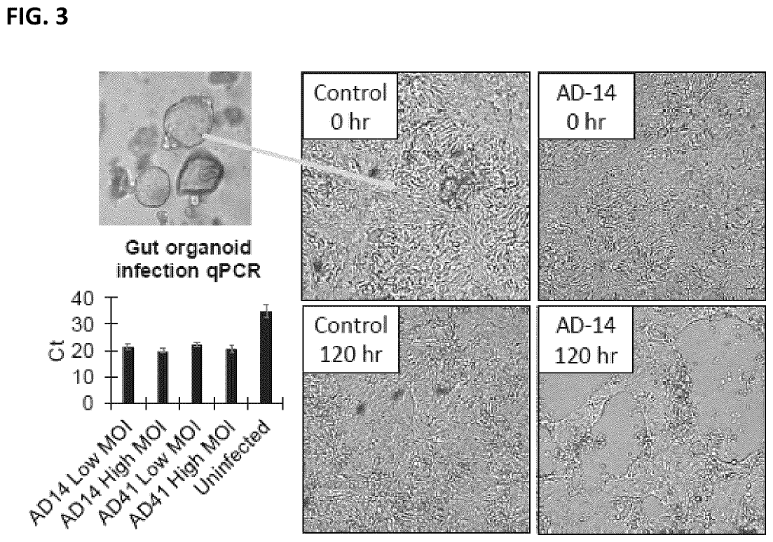

Plated and differentiated gut organoids were infected with 2 strains of adenovirus (Ad-14, Ad-41) (FIG. 3) and influenza A virus. Timelapse brightfield imaging was used to collect data to train a computer vision model to differentiate infected and uninfected tissues.

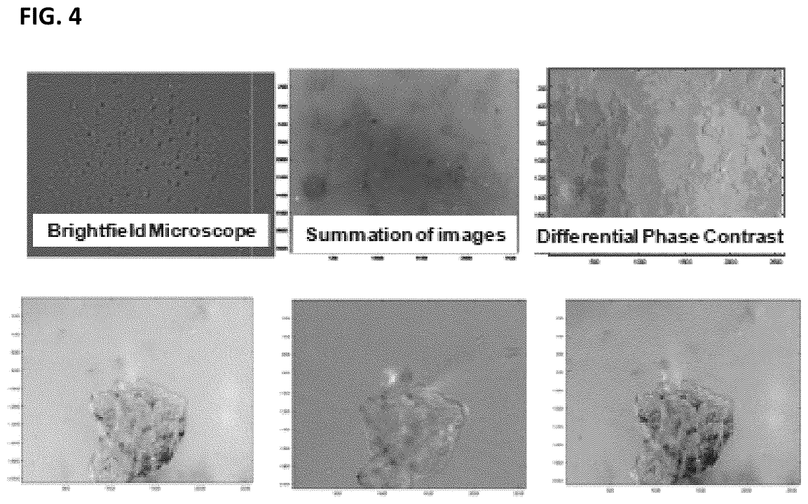

Quantitative Phase Imaging-Microscope Build: Quantitative phase imaging (QPI) works by asymmetrically illuminating a sample with different wavelengths of light, creating images with different phase paths. Integrating these images together creates a differential phase contrast image (FIG. 4). Using a back end software algorithm refractive index (RI) changes may be measured, even in thick organoid samples. Since infection triggers changes in cellular chemical composition, this should change the RI and cause a phase shift that may be measured using QPI.

### Example 2

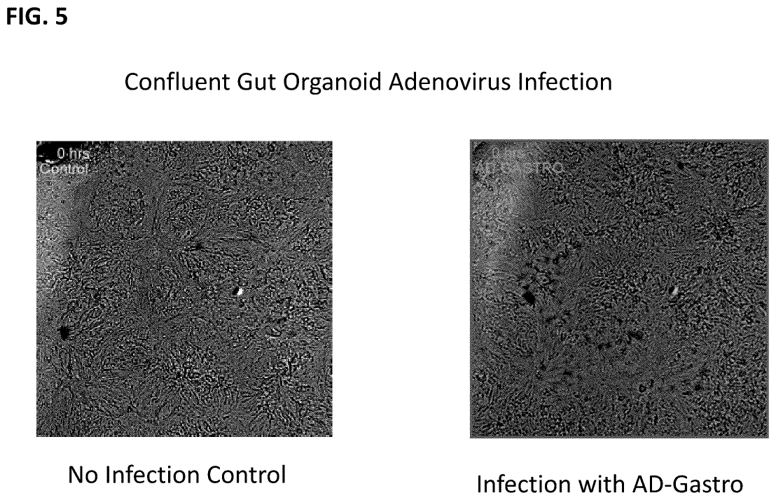

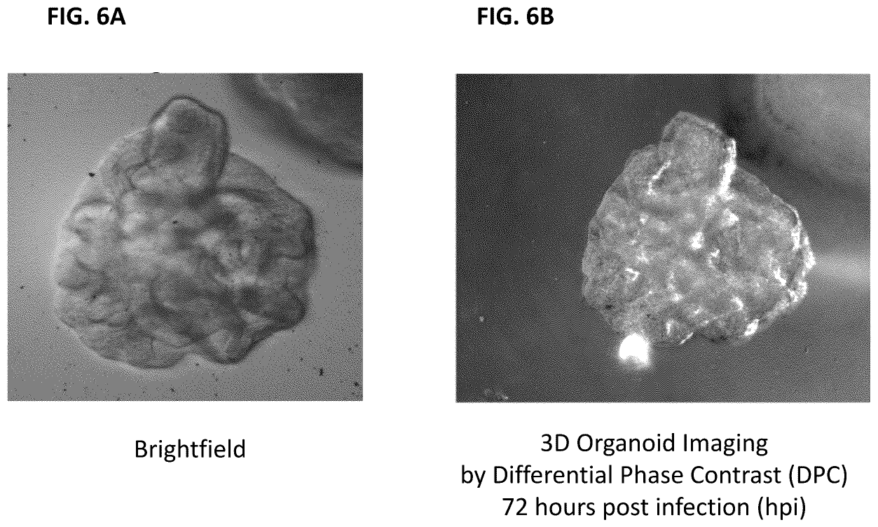

Confluent gut organoids in cell culture were infected with adenovirus, or not infected as a control (FIG. 5). Bright field and differential phase contrast images of an infected organoid are shown in FIGS. 6A and 6B. The images taken were 72 hours after infection.

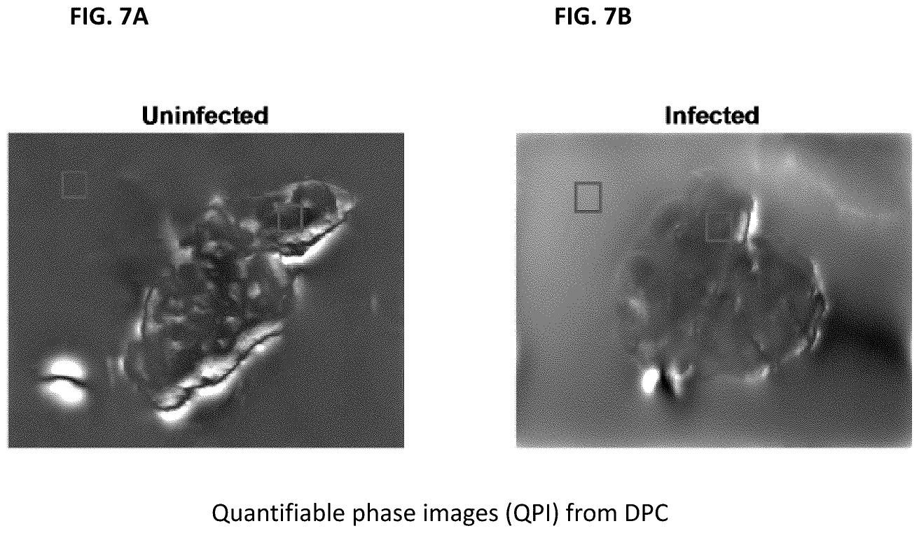

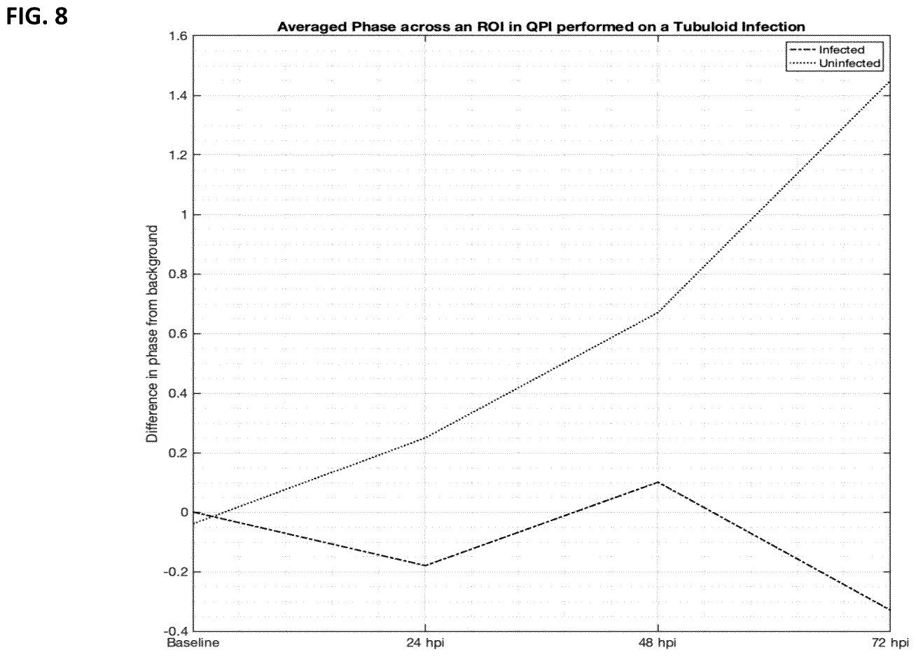

Determining changes between uninfected and infected organoids, regions of interest (ROIs) were taken of background regions and organoid regions over time and averaged (FIGS. 7A and 7B). Averaged phase across an ROI of an infected and uninfected tubuloid are shown in FIG. 8. The difference in phase from background is determined at 24 hours, 48 hours, and 72 hours after infection. Uninfected and infected organoids show significant difference over time, representing the change of more or more characteristics of the infected organoid.

### Exemplary Computer System

FIG. 10 depicts an example computer apparatus 1200 for use with one or more embodiments described herein.

As an example, apparatus 1200 may be a computer to implement certain techniques disclosed herein, such as a computing device to implement the process flows of one or more of FIGS. 9A and 9B. As an example, the steps in the methods illustrated in one or more of FIGS. 9A and 9B may be performed by one, two, three, four, or more apparatuses 1200. As an example, apparatus 1200 may be a smartphone or other portable computer device (e.g., a tablet or a laptop), a personal computer, or the like, to perform the steps in the methods described in one or more of FIGS. 9A and 9B.

Apparatus 1200 may include one or more processors 1202, one or more memory 1203, one or more input devices 1205, and one or more output devices 1206. In some embodiments, apparatus 1200 may be a computer that includes a web browser or a software application.

Input to apparatus 1200 may be provided by one or more input devices 1205, provided from one or more input devices in communication with apparatus 1200 via link 1201 (e.g., a wired link or a wireless link; e.g., with a direct connection or over a network), and/or provided from another computer(s) in communication with apparatus 1200 via link 1201. Link 1201 may provide a network interface to other computing devices via a network, such as the Internet, a local area network (LAN), a wide area network (WAN), a cellular phone network or hot spot, a cable modem, or the like. Link 1201 may include, for example, a wired or wireless network adapter and/or a wireless data transceiver for use with a mobile telecommunications network. Input device 1205 may include, for example, a keyboard, a touchpad, a keypad, a pointing device, a mouse, a stylus, a touch sensitive panel (e.g., a touch pad or a touch screen), a biometric input device, a mixed-reality headset, an audio input device, a visible light camera, and/or an infrared camera.

Output for apparatus 1200 may be provided by one or more output devices 1206, provided to one or more output devices in communication with apparatus 1200 via link 1201, and/or provided from another computer(s) in communication with apparatus 1200 via link 1201. Output device 1206 may include, for example, a display, a mixed-reality headset, one or more individual LEDs, and/or a speaker.

In some embodiments, one or more input devices 1205 and one or more output devices 1206 may be combined into one or more unitary input/output devices (e.g., a touch screen on a smartphone or tablet PC).

In some embodiments, based on input from one or more input devices 1205 or input from outside apparatus 1200 via link 1201, one or more processors 1202 may perform operations as described herein. As an example, user input may be received from one or more input devices 1205. As an example, input may be from another computer in communication with apparatus 1200 via link 1201. As an example, input may be from one or more input devices in communication with apparatus 1200 via link 1201.

In some embodiments, one or more processors 1202 may perform operations as described herein and provide results of the operations as output. As an example, output may be provided to one or more output devices 1206. As an example, output may be provided to another computer in communication with apparatus 1200 via link 1201. As an example, output may be provided to one or more output devices in communication with apparatus 1200 via link 1201. An output device may include a display or screen to present a graphical user interface (e.g., a web browser and/or a client application) to a user.

Memory 1203 may be accessible by one or more processors 1202 so that one or more processors 1202 may read information from and write information to memory 1203. Memory 1203 may store instructions that, when executed by one or more processors 1202, implement one or more embodiments described herein. Memory 1203 may be a non-transitory computer readable medium (or a non-transitory processor readable medium) containing a set of instructions thereon for identifying and characterizing pathogens, wherein when executed by a processor (such as one or more processors 1202), the instructions cause the processor to perform one or more methods discussed herein, such as, for example, the methods of FIGS. 9A and 9B. As an example, apparatus 1200 may be a smartphone, and memory of the smartphone may store an application, or app, to perform embodiments described herein.

Apparatus 1200 may include: one or more processors (such as one or more processors 1202); and memory (such as memory 1203) accessible by the one or more processors, the memory storing instructions that when executed by the one or more processors, cause the apparatus to perform one or more methods described herein. As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

Memory 1203 may be a non-transitory processor readable medium containing a set of instructions thereon, wherein when executed by one or more processors (such as one or more processors 1202), the instructions cause the one or more processors to perform one or more methods described herein. In some embodiments, memory 1203 may be a local storage device. Additionally, or alternatively, memory 1203 may synchronize with or access data from a remote storage location, such as a cloud storage device. In some cases, memory 1203 may include a database. The term “non-transitory,” as used herein, is a limitation of the medium itself (i.e., tangible, not a signal) as opposed to a limitation on data storage persistency (e.g., RAM vs. ROM).

### Additional Considerations

A neural network (NN) may be described as a computation graph consisting of several layers of computation. Each layer may consist of one or more units, where each unit performs an elementary computation. A unit may be connected to one or more other units, and the connection may be associated with a weight. The weight may be used for scaling the signal passing through the associated connection. Weights may be learnable parameters, i.e., values which may be learned from training data. There may be other learnable parameters, such as those of batch-normalization layers.

In some neural networks, such as convolutional neural networks for image classification, initial layers (those close to the input data) may extract semantically low-level features such as edges and textures in images, whereas intermediate layers extract more high-level features. After the feature extraction layers, there may be one or more layers performing a certain task, such as classification, semantic segmentation, object detection, denoising, style transfer, super-resolution, etc. One property of neural networks (and other machine learning methods or tools) is that neural networks are able to learn properties from input data, e.g., in supervised way or in unsupervised way. Such learning may be a result of a training algorithm, or of a meta-level neural network providing the training signal.

In some embodiments, the training algorithm may include changing some properties of the neural network so that the output of the neural network is as close as possible to a desired output. For example, in the case of classification of objects in images, the output of the neural network may be used to derive a class or category index which may indicate the class or category that the object in the input image belongs to. Training may occur by minimizing or decreasing the output's error, also referred to as the loss or loss function. Examples of losses include mean squared error, cross-entropy, etc. In some embodiments using deep learning techniques, training may be an iterative process, where at each iteration the algorithm may modify the weights of the neural network to make a gradual improvement of the neural network's output, i.e., to gradually decrease the loss, by means of, for example, a gradient descent technique. For example, at each training iteration, gradients of the loss function with respect to one or more weights or parameters of the neural network may be computed, for example by a backpropagation technique; the computed gradients may then be used by an optimization routine, such as Adam or Stochastic Gradient Descent (SGD), to obtain an update to the one or more weights or parameters. The weights of a neural network may sometimes be referred to as learnable parameters or simply as parameters.

Training a neural network may be an optimization process, but a final goal of the training may be different from mere optimization. In optimization, a goal may be to minimize a function. In machine learning, a goal of the training process may be to make the model learn at least some properties of the data distribution based on a limited training dataset. In other words, the goal may be to learn to use a limited training dataset in order to learn to generalize to previously unseen data, i.e., data which was not used for training the model. This may be referred to as generalization. In some embodiments, data may be split into at least two sets, the training set and the validation set. The training set may be used for training the network, i.e., to modify the adjustable or learnable parameters of the neural network in order to minimize the loss. The validation set may be used for checking the performance of the neural network on data which was not used to minimize the loss, as an indication of the final performance of the neural network. In some embodiments, the errors on the training set and on the validation set may be monitored during the training process to understand the following, for example: (i) if the neural network is learning at all—in this case, the training set error should decrease, otherwise the model may be underfitting the training data; and/or (ii) if the neural network is learning to generalize—in this case, also the validation set error needs to decrease and to be not too much higher than the training set error. If the training set error is low, but the validation set error is much higher than the training set error, or the validation set error does not decrease, or the validation set error even increases, the neural network may be overfitting the training data. If the neural network is overfitting the training data, the neural network may have memorized the training set's properties and may perform well only on the training set, but may perform poorly on a set not used for training the parameters of the neural network.

The term generative artificial intelligence (AI), or generative modeling, or generative machine learning (and other similar terms), may refer to a class of models learned from data that are capable of generating new data. For example, a generative model may extrapolate new data samples based on a learned data distribution. Examples of tasks or purposes that a generative model may perform or achieve include, but are limited to, the following: visual spatial extrapolation (e.g., spatially extending a picture), visual temporal extrapolation (e.g., generating new pictures based on other pictures in a video, such as future pictures based on past pictures). State-of-the-art generative models may be based on neural networks. At least some of the basic components or layers of a generative neural networks may be similar or same as those of a non-generative neural networks. Example of such components are convolution layers, non-linear layers, fully-connected layers, normalization layers, attention layers, etc.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method of evaluating an effect of at least one agent on an organoid, the method comprising:
providing the organoid in a cell culture or microfluidic platform;
contacting the organoid with at least one agent;
acquiring a plurality of images of the organoid and a control organoid;
analyzing the plurality of images for a change of at least one characteristic of the organoid, wherein the change of the at least one characteristic is indicative of a disease or a disorder, wherein the analyzing comprises comparing the organoid to the control organoid to determine the change of the at least one characteristic; and
assessing the effect of the at least one agent at least in part on the change of the at least one characteristic of the organoid.

2. The method of claim 1, wherein the at least one agent comprises:
a pathogen,
a chemical agent, and/or
a biological agent.

3. The method of claim 1, wherein the plurality of images of the organoid and a control organoid are acquired with quantitative phase imaging (QPI).

4. The method of claim 1, wherein assessing the effect of the at least one agent comprises comparing the change of the at least one characteristic of the organoid to the control organoid.

5. The method of claim 1, wherein the control organoid comprises an organoid not contacted with at least one agent.

6. The method of claim 1, wherein the control organoid comprises an organoid contacted with at least one agent.

7. The method of claim 1, wherein the analyzing further comprises comparing a refractive index from the plurality of images of the organoid and/or the control organoid for at least 1 hour after contacting the organoid with at least one agent.

8. The method of claim 7, wherein the analyzing is for about 12 hours, about 24 hours, about 36 hours, about 48 hours, about 60 hours, about 3 days, about 4 days, about 5 days, about 6 days, about 1 week, about 2 weeks, or continuously after contacting the organoid with at least one agent.

9. The method of claim 1, wherein the at least one characteristic comprises size of the organoid and/or swelling of cells of the organoid.

10. The method of claim 9, wherein the organoid decreases, stays the same, or increases in size compared to prior to contacting the organoid with the at least one agent.

11. The method of claim 9, wherein swelling of cells of the organoid increases compared to prior to contacting the organoid with the at least one agent.

12. The method of claim 1, wherein the effect of the at least one agent is determined with at least one neural network configured to predict the effect of the at least one agent when provided with the plurality of images of the organoid and the control organoid.

13. A method for identifying a therapeutic agent for preventing or treating a disease or disorder, the method comprising:
providing a diseased organoid in a cell culture or microfluidic platform;
administering the therapeutic agent to the diseased organoid generating a treated organoid;
acquiring a plurality of images of the treated organoid and a control organoid;
analyzing the plurality of images for a change of at least one characteristic of the treated organoid, wherein the change of the at least one characteristic is indicative of the suitability of the therapeutic agent for preventing or treating the disease or disorder, wherein the analyzing comprises comparing the treated organoid to the control organoid to determine the change of the at least one characteristic; and
identifying a candidate therapeutic agent for preventing or treating the disease or disorder at least in part based on the change of the at least one characteristic of the organoid.

14. The method of claim 13, wherein the plurality of images of the treated organoid and the control organoid are acquired with quantitative phase imaging (QPI).

15. The method of claim 13, wherein the diseased organoid comprises an organoid infected with a pathogen.

16. The method of claim 15, wherein the control organoid comprises:
an organoid not infected with the pathogen;
an organoid not infected with the pathogen and treated with the therapeutic agent; and/or
an organoid infected with the pathogen and not treated with the therapeutic agent.

17. The method of claim 13, wherein the diseased organoid comprises an organoid comprising cells having the disease or disorder.

18. The method of claim 17, wherein the control organoid comprises:
an organoid comprising healthy cells that do not have the disease or disorder;
an organoid comprising healthy cells that do not have the disease or disorder and treated with the therapeutic agent; and/or
an organoid comprising cells having the disease or disorder and not treated with the therapeutic agent.

19. The method of claim 13, wherein the analyzing further comprises comparing a refractive index from the plurality of images of the treated organoid and/or the control organoid for at least 1 hour after administering the therapeutic agent to the diseased organoid.

20. The method of claim 19, wherein the analyzing is for about 12 hours, about 24 hours, about 36 hours, about 48 hours, about 60 hours, about 3 days, about 4 days, about 5 days, about 6 days, about 1 week, about 2 weeks, or continuously after administering the therapeutic agent to the diseased organoid.

21. The method of claim 13, wherein identifying a candidate therapeutic agent comprises assessing an effect of the therapeutic agent on the diseased organoid by comparing the change of the at least one characteristic of the treated organoid to the control organoid.

22. The method of claim 21, wherein the at least one characteristic comprises size of the treated organoid and/or swelling of cells of the treated organoid.

23. The method of claim 22, wherein the treated organoid decreases, stays the same, or increases in size compared to prior administering the therapeutic agent to the diseased organoid.

24. The method of claim 22, wherein swelling of cells of the treated organoid increases compared to prior administering the therapeutic agent to the diseased organoid.

25. The method of claim 13, wherein the suitability of the therapeutic agent for preventing or treating the disease or disorder is determined with at least one neural network configured to predict an effect of the therapeutic agent when provided with the plurality of images of the treated organoid and the control organoid.
